(19) United States
(12) Patent Application Publication
(10) Pub. No.: US 2026/0008987 A1
(43) Pub. Date: Jan. 8, 2026

(54) CELLULOSE-BASED MICROBIOLOGICAL CULTURE DEVICE

(71) Applicants: ASSOCIAÇÃO ALMASCIENCE - INVESTIGAÇÃO E DESENVOLVIMENTO EM CELULOSE PARA APLICAÇÕES INTELIGENTES E, CAPARICA (PT); UNIVERSIDADE NOVA DE LISBOA, LISBOA (PT); RAIZ - INSTITUTO DE INVESTIGAÇÃO DA FLORESTA E PAPEL, EIXO (PT); CLARA SAÚDE, LDA, PINHAL NOVO (PT)

(72) Inventors: Bruno Miguel RIBEIRO VEIGAS, ALHANDRA (PT); Bruno Brisolla RAVANELLO, AVEIRO (PT); Fausto Daniel DOS SANTOS QUEDA, LISBOA (PT); Catarina ROSA FERNANDES, LISBOA (PT); Bruna Alexandra OLIVEIRA RAMOS, PINHAL NOVO (PT); Sónia Isabel TORRES ALVES, MACIEIRA DE RATES (PT); Rita Helena DUARTE BAPTISTA, ALMADA (PT); Marta Helga FREIRE LOPES, EIXO (PT); Rodrigo FERRÃO DE PAIVA MARTINS, CAPARICA (PT); Elvira Maria CORREIA FORTUNATO, CAPARICA (PT); Carlos José CLARA DOS SANTOS, PINHAL NOVO (PT); André Filipe CARDOSO CLARA DOS SANTOS, PINHAL NOVO (PT)

(21) Appl. No.: 18/992,830

(22) PCT Filed: Jul. 11, 2023
(86) PCT No.: PCT/IB2023/057091

§ 371 Date: Jan. 9, 2025

(30) Foreign Application Priority Data
Jul. 11, 2022 (PT) 118095

(51) Int. Cl. C12M 1/12; C12M 3/06

(52) U.S. Cl. CPC C12M 25/04; C12M 23/16; C12M 37/00

(57) ABSTRACT

A cellulose-based microbiological culture device of bio-based and biodegradable materials, comprising a high-water absorbent cellulose layer with a hydrogel formulation comprising hydrocolloids that form a hydrogel upon rehydration. The present application further discloses a cellulose-based microbiological culture device with an integrated antibiogram or a standalone e-test device. The presently disclosed device is renewable, low-cost, has an increased durability and allows the reduction of laboratory waste.

### TECHNICAL FIELD

This application relates to a cellulose-based microbiological culture device for cell culture and a method to produce the same.

### BACKGROUND ART

Clinical microbiology is one of the key areas in clinical analysis laboratories, Used routinely for the identification and characterization of microorganisms in biological samples, it is a valuable tool for the therapeutic decision of clinicians, as well as the basis for important epidemiological studies related to infectious diseases, Bacterial culture remains the gold standard technique in clinical microbiological contamination assessment. This approach also enables the study of the antibiotic susceptibility of bacteria establishing recommendations for effective treatment. Clinical microbiology was revolutionized by the invention of the solid culture media in a Petri dish and, consequently, the identification of bacterial species in pure culture. Agar is the most common gelling agent used in the production of solid culture media. However, most of these have low shelf life (weeks) and require refrigeration during transport and storage (Lagier et al., 2015).

Document US2020248119 discloses a microbiological culture device including: a part made of absorbent material having an at least substantially planar upper face and incorporating into its thickness a dehydrated culture medium composition, resting on the part made of absorbent material, a sheet of dehydrated polysaccharide hydrogel which can be rehydrated. In the present invention the absorbent component is a fibrous cellulosic material where the hydrogel is applied over, in a way that the hydrogel is partially integrated into the fibre matrix, promoting adhesion between both layers. Additionally, herein the hydrogel composition is different from Document US2020248119, the growth media is incorporated on both the cellulosic fibrous material and the hydrogel that is composed of a membrane of dehydrated carboxy-methyl cellulose, pectin, sodium alginate and/or a mixture thereof. Specifically on ionic crosslinking the present invention states an interval of 40 to 120 g polysaccharides per litre of water outside of the claimed range of the said patent. Also, the present invention allows for the use of an ionic or covalent crosslinker, which grants the hydrogel the necessary structural characteristics for standard inoculation protocols.

Document WO2014013089 discloses a method for isolating at least one microorganism from a sample likely to be contaminated by said microorganism. The present invention differs from this document since it provides a technology that may or may not be integrated into a stand-alone microbial detection platform. The proposed technology may be used as a growth matrix using a standard plastic/glass petri dish or integrated as a stand-alone device. On the said integration, this patent states that there is always a layer comprising culture medium, however our technology/invention allows to be used as a blank growth matrix without any growth media; in this specific case the hydration would be done with the desired growth media instead of water; (this approach allows for this technology to be used with media that are not dehydratable such as blood based media for the detection of hemolytic bacteria or to be used for alternative inoculation approaches by using the growth matrix as a sample collection device). This hydration is made through a specific layer allowing for fast water absorption and homogeneous distribution while pushing the nutrients homogeneously distributed on the growth matrix onto the surface of said device.

Additionally, the provided integrations are different and new when compared to the said patent. The proposed integrations lead to the increase of device functionality such as complete antibiogram analysis with several antibiotics and provided predesigned halos to facilitate sample scoring, and the integration with gradients of specific antibiotics in an all-in-one solution for the detection of minimum inhibitory concentration (MIC) leading to a full characterization of antibiotic susceptibility. None of these integrations are described in the above-mentioned patent. Additionally, the architecture of the device is different. In the description is said that the absorbent material and hydrogel are two mechanical separate materials, in the technology described herein there is partial integration of hydrogel onto the cellulosic fibrous material, leading to an effective adhesion between both materials. This approach allows the hydrogel to have better mechanical characteristics and lower deformation over rehydration cycles.

According to a preferred embodiment of said patent the volume of sample deposited on the culture medium is between 10 and 1000 μL. This document technology allows for a wider interval of inoculation volumes, thus increasing the spectrum of usage scenarios (1 to 5000 μL for a dish of 9 cm in diameter).

Document WO2015107228 discloses a device for culturing and/or isolating and/or detecting and/or identifying and/or counting at least one target microorganism in a sample. The present invention differs from this document in the sense that according to a preferred embodiment, the porous layer is opaque so as not to allow light from the fibrous material to pass. The device comprises a first porous layer, in contact with the fibrous substrate, comprising titanium dioxide and a second outer porous layer comprising kaolin. The cellulosic fibrous material used in the present invention is translucid when used with direct illumination; meaning that it does allow light to pass. Thus, no extra components material or layers are added to prevent light transmission.

The invention also relates to a support for the growth of a microorganism comprising at least one pigment, preferably inorganic chosen from the following list: kaolin, talc, titanium dioxide, calcium carbonate; none of which are used in the formulations from this invention. Additionally, the binder is styrene butadiene latex and/or styrene acrylic latex and/or methyl cellulose, and the crosslinking agent is selected from the following cross-linking agents: isocyanate and melamine formaldehyde. Again, the binder and cross-linking agents are different and there is no use of any of the above-mentioned materials. The referred patent uses only covalent crosslinking approaches via isocyanate and melamine-formaldehyde. The present invention uses both ionic or covalent crosslinking, where in the case of covalent crosslinking different crosslinkers are employed.

U.S. Pat. No. 6,756,225 B2 discloses thin film culture devices, as well as methods for harvesting cells from colonies on the culture device based on location of colonies on the device relative to the positioning structures, and a computer readable medium encoded with a computer program that identifies the position of colonies relative to the positioning structures. The present invention differs from this document in the sense that part of the hydrogel is integrated into the fibre substrate promoting adhesion and an interface between hydrogel and fibre layers. Additionally, in the present invention the fibrous substrate is part of the developed solution, being fundamental for maintaining the desired water content during incubation, assisting in the diffusion of the culture media, and serving as dispersion substrate for the crosslinker. Moreover, in this invention the fibrous and hydrogel layers have a higher grammage and consequently higher water retention, allowing the incubation for longer periods of time, while also allowing the use of an inoculation loop.

U.S. Pat. No. 8,753,834 B2 discloses an article for the detection of a microorganism in a liquid sample. The article comprises a microporous membrane and a barrier layer to selectively regulate the contact between the sample and a detection reagent. The present invention is similar to the previously presented one (U.S. Pat. No. 6,756,225 B2). The difference being the inclusion of a second detection device separated by a porous membrane and a barrier layer. This leads to a two-step protocol approach; after the first incubation period, there is the removal of the barrier layer between the devices and the diffusion of the solution through the porous membrane to the second detection device, where a detection agent is incorporated. The differences in architecture and materials from doc U.S. Pat. No. 6,756,225 B2 stand. Additionally, the proposed method herein allows for the use of standard protocols without the need of a two-stage process while reducing the analysis time, since it uses only one incubation period for no more than 72 hours.

Document WO 2021/170606 A1 discloses a ready-to-use device and method for testing differential growth of microorganisms. The device comprises a base sheet, a dry medium layer on top of the base sheet, a water vapor impermeable lid sheet to cover the medium layer and one or more differentiating agents are positioned under, within, on and/or above the dry medium layer in a spatially resolved manner. In the present invention an ionic or covalent crosslinker is used to yield hydrogels with the necessary rigidity which allows the use of an inoculation loop and the microbial growth on the surface of the detection device. Moreover, the present invention uses a different architecture and includes colorimetric detection of cell growth to facilitate the detection and characterization of minimum inhibitory concentration of bacterial samples. Herein, the device is designed so that the result is similar to those attained in standard methods with the bacteria creating a halo around the antibiotic, which in this case can be printed to define what is the threshold for sample scoring.

Recent studies have demonstrated the use of impregnated paper media in the culture of cells (Ng et al., 2017). Very few have explored the use of paper-based media in microorganism culture and have constrained their studies to chromogenic testing (Funes-Huacca et al., 2012; Deiss et al., 2013; Hol et al., 2019; He et al., 2020). Paper is an economical, abundant, biocompatible, and recyclable biomaterial that may reduce the overall cost of commercial culture media used in laboratory practice. In addition, cellulose-based hydrogels can substitute agar medium with the advantage of being able to dehydrate and subsequently rehydrate without losing structural properties, allowing it to be stored in a dry state and dramatically increase the shelf life of these products. Previous published studies on paper-based media have only covered controlled samples and had limited experimental data, making it impossible to be validated and certified by regulatory standards (Kelvin et al., 2017; Deiss et al., 2014).

The agar media generally used in laboratory practice for microbiological contamination assessment and are already packed hydrated require refrigerated conditions during transport and storage. A culture medium that can be dried or lyophilized can have its shelf life extended from weeks to months. The increased shelf-life complemented with fewer storage requirements (e.g. humidity and temperature control) would allow the use of this microbial culture device in peripheral laboratories and/or resource-poor settings where dedicated infrastructure is not present. In addition, the reduction of laboratory waste would result in obvious environmental advantages and reduced costs.

A solution currently available is the Petrifilm plate (document U.S. Pat. No. 6,638,755 B1). It is heavily used by microbiology-related industries and is meant to be more efficient than conventional plating techniques. Ingredients usually vary, but generally a Petrifilm comprises a cold-water soluble gelling agent such as guar gum, xanthan gum, alginate, carboxymethyl cellulose, hydroxyethyl cellulose, etc., nutrients, and a chromophore indicator specific for the strain to be detected. Dry media plates have become widely used because of their cost-effectiveness, simplicity, convenience, and ease of use. Since they are thin (similar to paper typical thickness), more plates can be stacked together than Petri dishes. The 3M patent portfolio related to the Petrifilm product is U.S. Pat. Nos. 6,756,225 B2, 8,753,834 B2, WO 2019/156259 A1 and WO 2005/024047 A2. Other alternatives focused on the same concept have been developed, a couple of which are worth mentioning: R-Biopharm Compact Dry™ and Liofilchem Easy Dry™. From the available dry culture media portfolio, only the last two are certified for room temperature storage (up to +25° C., 24 months), while the competitor products require storage temperature below 8° C. However, none of these products lend themselves to the use of an inoculation loop and isolation techniques carried out by streaking, due to the irregular surface of the culture medium.

As far as antibiotic susceptibility tested is concerned, two major tests are accepted as the ‘go to’ when antibiotic resistance analysis are required: antibiograms for a general screening, and e-Tests for a specific antibiotic assay. Antibiograms are usually commercialized as impregnated small dishes that are posteriorly manually integrated in the previously mentioned agar petri dishes and discarded. E-Tests work similarly, requiring a whole cultured dish for a single analysis.

Susceptibility tests as alternatives of the current market gold standard have also been protected by patents: EP0157071; U.S. Pat. No. 4,778,758, where stripes with varying concentrations of growth inhibitory reagents are described. As far as the materials used, paper substrates have been used and previously patented (WO2011/032683; EP2480682B1). Ink jet technique for the antibiotic concentration gradient deposition has also been disclosed in EP102251018, referring to individual antibiogram dishes. Some processing methods have been patented already, related to automated and direct result analysis. Namely, in U.S. Pat. No. 9,435,789B2; EP1076098A1 and EP2235203B1 present protected procedures for the treatment of the results for MIC determination tests and for gas-producing bacteria, respectively, using specific individual devices for cell growth.

In what concerns the eTest-based antibiotic susceptibility testing, the original concept of a gradient strip for a precise determination of the minimum inhibitory concentration (MIC) of antibiotic molecules was secured via EP0157071. The MIC Evaluator (MICE) is based on the same principle as the afore mentioned patent, using a polymer strip graded with a concentration scale of numerous clinically or biologically active substances. Both in the patent EP0157071 and for the MIC Evaluator, the carriers are made of plastic. Other earlier patents, such as patent U.S. Pat. No. 4,778,758, describe and claim a device for the determination of microorganisms consisting of a sealed rectangular enclosure containing a transparent non-porous strip for the test. This patent claims “a rectangular, transparent, non-porous, inert test strip” made of an “inert polymeric material selected from the group consisting of acrylic plastic material, styrene plastic material and vinyl chloride plastic material”. In 2011 this concept was disclosed with impregnated paper strip in WO 2011032683. Additionally, the inkjet approach for the production process of such devices is also protected in documents EP 102251018 A and CN102251018A.

### SUMMARY

The present invention relates to a cellulose-based microbiological culture device comprising:
a dehydrated hydrogel coating layer (1) arranged on top of or partially integrated on at least one cellulosic fibrous layer (2); and
at least one rehydration means (3);
wherein the dehydrated hydrogel coating layer (1) comprises:
at least one binding agent from the family of cellulose-derived hydrocolloids in a concentration between 0.1 and 6% wt.;
at least one gelling agent from the family of polysaccharide hydrocolloids in a concentration between 0.1 and 68 wt.; and
a plasticizer in a concentration between 1 and 15% wt.;
   and wherein the at least one cellulosic fibrous layer (2) further comprises a cross-linker agent in a concentration between 0.01% to 5% wt.

In one embodiment the device comprises an interface (1.1) formed by the hydrogel layer (1) being partially integrated in the cellulosic fibrous layer (2).

In one embodiment the dehydrated hydrogel coating layer (1) has a thickness between 5 and 500 μm.

In one embodiment the device comprises at least two stacked cellulosic fibrous layers (2).

In one embodiment the hydrocolloid binding agent is selected from the group comprising cellulose and/or a cellulose derivative, a polynucleotide, a polypeptide, a polysaccharide, a natural rubber, a polyphenolic polymer, a complex of polymers of large chain fatty acids or their mixtures or their composites.

In one embodiment the hydrocolloid binding agent is selected from sodium carboxymethyl cellulose (CMC), carboxyethyl cellulose (CEC), hydroxypropyl cellulose (HPC), hydroxyethyl cellulose (HEC), methyl cellulose (MC), ethyl cellulose (EC), hydroxypropylmethyl cellulose (HPMC), hydroxyethylmethyl cellulose (HEMC), hydroxyethylpropyl cellulose (HEPC), bacterial cellulose (BC), cellulose nanofiber (CNF), cellulose nanocrystals (CNC), microfibrillated cellulose (MFC), hydroxypropyl methylcellulose phthalate, or their mixtures.

In one embodiment the gelling agent from the family of polysaccharide hydrocolloids is selected from sodium alginate, gellan gum, carrageenan, guar gum, xanthan gum, locust bean gum, gum arabic, pectin, or modified starch.

In one embodiment the plasticizer is selected from polyvinyl alcohol (PVOH), ethylene glycol (EG), diethylene glycol (DEG), triethylene glycol (TEG), polyethylene glycol (PEG), propylene glycol (PG), glycerol, erythritol, sorbitol, mannitol, maltitol, xylitol, polyols, fatty acids, or vegetal oils.

In one embodiment the cellulosic fibrous layer (2) comprises cellulose fibers with weight in the range of 50 to 500 g/m², selected from bleached softwood fibers, unbleached softwood fibers, bleached hardwood fibers, unbleached hardwood fibers, cotton fibers or their mixtures.

In one embodiment the cross-linker agent is selected from zinc chloride, magnesium chloride, copper sulfate, nickel sulfate, calcium chloride, epichlorohydrin, or citric acid.

In one embodiment the cellulosic fibrous layer (2) further comprises a culture medium.

In one embodiment the cellulosic fibrous layer (2) further comprises a wet strength agent selected from polyaminopolyamide-epichlorohydrin resins, glyoxalated polyacrylamide resins, polyvinylamine resins, polyethylenimine resins, polyisocyanate resins, dialdehyde starch, or a mixture thereof.

In one embodiment the cellulosic fibrous layer (2) further comprises a colorimetric agent or cell growth markers.

In one embodiment at least one rehydration means (3) is selected from a layer arranged below the cellulosic fibrous layer (2), a perforation in the hydrogel coating layer (1) and the cellulosic fibrous layer (2), a perforation on the hydrogel coating layer (1), or a lateral access tab via one hydration coating layer (1), or a rehydration tab, micro perforation of selected areas of the device, or a single use blister.

In one embodiment the cellulosic fibrous layer (2) comprises at least one threshold halo (5) for an antibiotic and the hydrogel coating layer (1) comprises at least one antibiotic solution (4) coincident with a least one threshold halo (5) forming an antibiogram.

In one embodiment the cellulosic fibrous layer (2) comprises a ruler like structure (7) and the hydrogel coating layer (1) comprises antibiotic gradient (6) aligned with the ruler like structure (7), forming an E-test.

In one embodiment, a double-sided adhesive material is arranged on the underside surface of the cellulosic fibrous layer (2).

The present invention also relates to a method of producing the cellulose-based microbiological culture device comprising the following steps:
impregnating a cross-linker agent in a cellulosic fibrous layer (2);
applying a hydrogel formulation in the cellulosic fibrous layer (2) forming at least one hydrogel layer (1);
drying the device at a temperature between 20 to 50° C.;
Optionally, adding a culture medium solution to the cellulosic fibrous layer (2) and drying the device at a temperature between 20 to 50° C.

Optionally, after applying the hydrogel formulation, the device can be washed with water for a period of time of less than 40 min and then dried at a temperature between 20 to 50° C.

In one embodiment the applying step is repeated according to the number of hydrogel coating layers (1) desired.

In one embodiment it further comprises a step of stacking and bonding at least two cellulosic fibrous layer (2) on top of each other.

In one embodiment it further comprises the steps of printing at least one area with an antibiotic solution in the hydrogel coating layer (1) and printing at least one threshold halo on the fibrous cellulosic layer (2) for at least one antibiotic.

In one embodiment it further comprises the steps printing at least an area with at least one antibiotic gradient in the hydrogel coating layer (1).

The present invention also relates to the use of the cellulose-based microbiological culture device for culture, detection, characterization, identification, and enumeration of microorganisms.

### General Description

The present invention aims to reduce the overall material waste in the diagnostic process, by using renewable, bio-based, low-cost materials. The main goal is to replace the commonly used agar with a cellulose-based solution comprising a hydrogel that can be easily rehydrated, suppressing the need of refrigeration storage and transportation in wide temperatures range, therefore increasing the product shelf-life. While rehydratable gels are already present on the market, most of the commercial solutions work with chromophores, meaning that each product is specific to a bacteria strain. Additionally, these products allow the microorganisms to grow inside the matrix of hydrogel, changing colony morphology and hindering post identification analysis protocols (e.g., antibiogram). The present invention can have generic applications in microbial contamination assessment, with the possibility of chromophore integration for specific cases.

The present invention is a cellulose-based microbiological culture device for application in the field of microbiological analysis. This technology envisions the substitution of agar products in clinical microbiological testing by a bio-based, biodegradable, and recyclable solution.

Two embodiments of the use of the present invention are proposed for antibiotic susceptibility testing, that can reduce assay time while providing user-friendly results, without the need of an associated reading hardware/software.

1) Antibiograms can be integrated in the cellulose-based microbiological culture device. Antibiotics are directly printed on the hydrogel coating and have resistant/susceptible visual indicators printed on the underlying fibrous cellulose material, allowing for a direct analysis, differentiated from what the current market offers. Delimitation areas can be directly printed in the underlying cellulose fibrous material, allowing for easier mask alignment for the antibiotic deposition. Halos can be printed based on the defined radius for each antibiotic for visual direct analysis, giving a quick resistant/susceptible result depending on the obtained halo size.

The incorporation of an antibiogram screening test in the cellulose-based microbiological culture device is innovative and reduces the analysis time dispended with ready-to-use prepared culture media. Also, the integrated test has threshold halos for each antibiotic making it easier to score each plate without any external equipment.

2) E-Tests can be integrated in the cellulose-based microbiological culture device to provide a colorimetric result where the minimum inhibitory concentration (MIC) can be directly determined, while suppressing the need of a whole culture petri dish, therefore reducing waste, time and costs while maintaining biodegradability. In this case, the presently disclosed device has a culture medium with an antibiotic gradient and a colorimetric agent that is suitable to facilitate reading microbial growth.

The proposed “E-test” solution is the first to integrate the antibiotic gradient, colorimetric detection, and culture medium in one device. Working with an integrated culture medium suppresses the need of a separate culture dish for this test, reducing waste material and analyst time, while providing a visual color-based direct result.

The present invention was developed to be an alternative to the gold standard agar products in clinical microbiological testing focusing on bio-based and renewable materials. Cellulose and its derivates are used as multifunctional materials for new generation solid culture media. Aiming at the development of a fully-fledged clinical microbiology contamination assessment platform, the specific characteristics of the technology disclosed herein are:
1—A solid culture medium based on cellulose and hydrogel that is rehydratable before use;
2—Increased shelf life of the culture medium;
3—Use of bio-based materials;
4—Integrated cellulose-based antibiogram test kit with cellulose-based culture medium and colorimetric cell growth detection.

With a lyophilized culture medium, the durability of the present invention can be increased from weeks to months. The increased shelf-life complemented with fewer storage requirements (e.g. humidity and temperature control) allows this technology to be used in resource-poor settings where the infrastructure is not present. In addition, the reduction of laboratory waste will result in obvious environmental advantages and reduced costs.

The invention described herein differs significantly from what is currently available, relying on an incorporated culture medium, a possible colorimetric detection system and a different production process.

Th present invention is developed on bio-based, renewable, and recyclable materials. These materials are extremely important since they lower the cost of waste management and are aligned with a sustainable and eco-friendly vision for this sector. In addition, it is possible to sterilize the used products and recycle cellulose into the production line, contributing to a zero-waste circular economy.

The present invention has a plethora of laboratory applications (e.g. Clinical, research and industrial such as the food and beverage industries) in the area of microbiology, even those that have residual activities. This invention is a support for microorganism growth different from gel the agarose currently used in microbiology laboratories around the world. The proposed substitution of materials will dramatically increase the shelf-life of these products, without requiring refrigeration during transport and storage, and allowing it to be implemented in markets where stable power supply is not guaranteed (e.g. underdeveloped and developing countries). This technology will contribute to better and affordable health care services all around the world. In addition, new microbiology laboratories with limited resources could have particular interest in the product because it does not require refrigeration during storage.

The present invention solves the problems of prior art by impregnating, or laminating, or depositing a hydrogel in a cellulosic fibrous layer, forming a layer zone impregnated with a hydrogel, which is loaded with growth medium in order to allow proper microorganism growth. The hydrogel is capable of multiple dehydration and rehydration cycles without loss of physical or chemical characteristics.

The present invention solves the problems of prior art regarding the growth of microorganisms on the matrix surface allowing the for implementation of standard post growth identification and characterization protocols. Other products in the market focus on the growth of microorganisms inside the gel-like material and identification is made via colorimetric detection. There is no colony morphology analysis nor post growth harvesting of colonies for post growth testing (e.g., antibiotic susceptibility testing, strain identification, etc.).

Another approach used by the present invention is the coupling of a cellulose-based fibrous layer and cross-linking agents. The use of this approach allows for the homogeneous distribution of the cross-linking agents, thus further improving the hydrogel characteristics. Additionally, the inclusion of the crosslinker agent in the fibrous layer creates a barrier like material that prevents the impregnation of the entire cellulose fibrous material with the hydrogel during application, allowing to maintain desired characteristics such as hydration rate and water retention capabilities.

The use of a cellulose fibrous layer as substrate also allows for faster hydration while increasing the availability of nutrient and the growth matrix surface. The underlining cellulose fibrous material is used as a passive fluidic structure that allows for a fast absorption of water. This characteristic makes the hydrogel to be homogeneously hydrated perpendicular to the surface pushing the nutrients to the surface.

Moreover, hydrogels are interesting materials for growth layers since their performance regarding optical and mechanical properties are easily tuned, so they can be flexible, dehydratable and biocompatible.

### BRIEF DESCRIPTION OF DRAWINGS

For an easier understanding of this application, figures are attached in the annex that represents the preferred forms of implementation which nevertheless are not intended to limit the technique disclosed herein.

FIG. 1 shows a cross-section of the layers of one embodiment of the cellulose-based microbiological culture device.

FIG. 2 shows a top view of the layers of one embodiment of the cellulose-based microbiological culture device.

FIG. 3 shows a scanning electron microscopy (SEM) image of the layers of one embodiment of the cellulose-based microbiological culture device.

FIG. 4 shows a SEM image of the layers of one embodiment of the cellulose-based microbiological culture device.

FIG. 5 shows a cross-section of the layers of one embodiment of the cellulose-based microbiological culture device.

FIG. 6 shows a top view of the layers of one embodiment of the cellulose-based microbiological culture device with an antibiogram.

FIG. 7 shows a top view of the layers of one embodiment of the cellulose-based microbiological culture device with an antibiogram.

FIG. 8a shows a cross-section of the dry cellulose-based microbiological culture device with an ionic cross linker.

FIG. 8b shows a cross-section of the wet cellulose-based microbiological culture device with an ionic cross linker.

FIG. 8c shows a cross-section of the dry cellulose-based microbiological culture device with a covalent cross linker.

FIG. 8d shows a cross-section of the wet cellulose-based microbiological culture device with a covalent cross linker.

FIG. 9a shows the cellulose-based microbiological culture device with cellulosic fibrous layer with Mueller Hinton culture medium with E. coli growth.

FIG. 9b shows the cellulose-based microbiological culture device without cellulosic fibrous layer with Mueller Hinton culture medium with E. coli growth.

FIG. 9c shows the cellulose-based microbiological culture device with cellulosic fibrous layer with MacConkey culture medium with E. coli growth.

FIG. 9d shows the cellulose-based microbiological culture device without cellulosic fibrous layer with MacConkey culture medium with E. coli growth.

FIG. 9e shows the cellulose-based microbiological culture device with cellulosic fibrous layer with Mueller Hinton culture medium with S. Aureus growth.

FIG. 9f shows the cellulose-based microbiological culture device without cellulosic fibrous layer with Mueller Hinton culture medium with S. Aureus growth.

FIG. 9g shows the cellulose-based microbiological culture device with cellulosic fibrous layer with MacConkey culture medium without S. Aureus growth.

FIG. 9h shows the cellulose-based microbiological culture device without cellulosic fibrous layer with MacConkey culture medium without S. Aureus growth.

FIGS. 10a and 10b shows the cellulose-based microbiological culture device with Mueller Hinton culture medium and environmental strain growth.

FIG. 11 shows the e-test architecture. The hydrogel coating layer is applied on a cellulosic fibrous layer in accordance with the present invention. An extra layer of a ruler like structure (7) is arranged in the cellulosic fibrous layer to facilitate the scoring of samples. A layer of pre-printed antibiotic is added to the hydrogel coating layer with a gradient of antibiotic concentration aligned with the previous ruler like structure.

### DESCRIPTION OF EMBODIMENTS

Now, preferred embodiments of the present application will be described in detail with reference to the annexed drawings. However, they are not intended to limit the scope of this application.

The present invention relates generally to the field of microbiological analysis. More specifically, the proposed invention relates to a cellulose-based microbiological culture device that comprises a high-water absorbent cellulose layer with a hydrogel formulation comprising hydrocolloids that form a hydrogel layer upon rehydration.

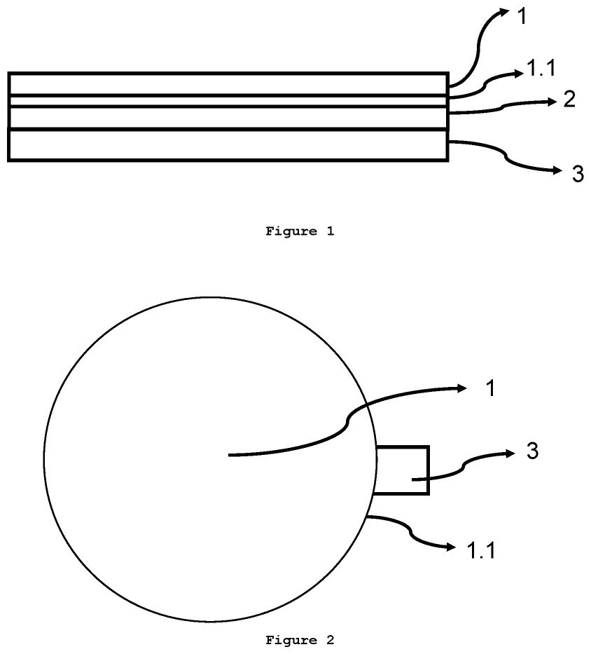

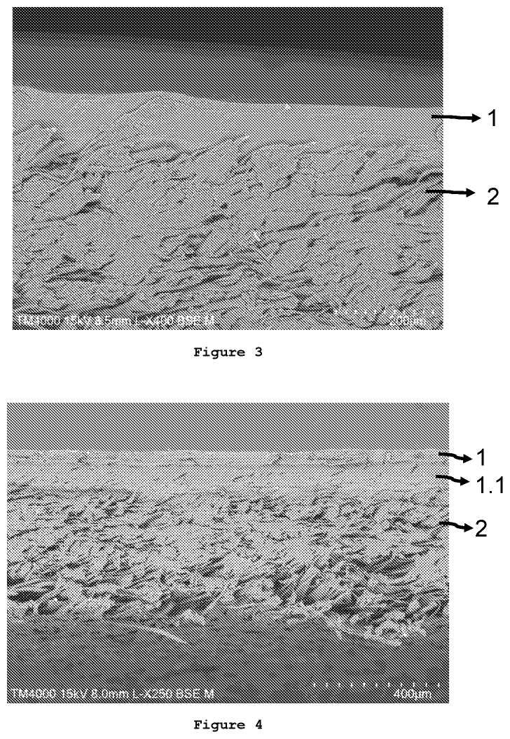

The cellulose-based microbiological culture device comprises the following layers as seen in FIGS. 1 to 5:
a dehydrated hydrogel coating layer (1), arranged on top of or partially integrated on at least one cellulosic fibrous layer (2); and
at least one rehydration means (3).

In the context of the present invention, “partially integrated on” means that a portion of the hydrogel layer (1) is impregnated in the cellulosic fibrous layer (2), creating a boundary section wherein the hydrogel material is mixed with the cellulosic fibrous material, forming an interface (1.1) shown in FIGS. 1, 2, 4 and 5. The interface (1.1) wherein the hydrogel material and the cellulosic fibrous material combine has a thickness between 0.1 and 500 μm. This partial integration assures a controlled expansion of the hydrogel in the desired direction upon hydration and prevents detachment of the hydrogel coating layer (1) from the cellulosic fibrous layer (2).

In the context of the present invention, “arranged on top of” means that the dehydrated hydrogel coating layer (1) is bound to the cellulosic fibrous layer (2) without creating an interface of combined materials.

### The Hydrogel Coating Layer (1):

The hydrogel coating layer (1) comprises:
at least one binding agent from the family of cellulose-derived hydrocolloids;
at least one gelling agent from the family of polysaccharide hydrocolloids;
a plasticizer.

In one embodiment, the number of layers of hydrogel (1) coating present on the cellulosic fibrous layer (2) varies from 1 to 4.

In one embodiment the dehydrated hydrogel coating layer (1) has a thickness between 5 and 500 μm, preferably between 20 and 200 μm.

In one embodiment the dehydrated hydrogel coating layer (1) has a basis weight (grammage) expressed in grams (g) per square meter (m²) between 20 and 600 g/m², preferably between 100 and 300 g/m².

This layer covers the pores in the cellulosic fibrous layer (2) to prevent microbial growth inside the fibrous structure.

In one embodiment, the hydrogel covers the entire surface of the cellulosic fibrous layer (2). In another embodiment, the hydrogel is placed only in specific areas allowing for the integration of antibiotic rich zones necessary to produce antibiogram and e-test devices.

In one embodiment the dehydrated hydrogel coating layer (1) further comprises at least one area with an antibiotic or at least one area with the standard concentration gradient of antibiotic.

Several hydrogel polymers may be selected according to their sustainable features regarding physical and chemical characteristics that promote the desired cellular growth while allowing for full dehydration and further rehydration steps.

The hydrocolloid binding agent for the hydrogel coating layer (1) is selected from the group comprising cellulose and/or a cellulose derivative, a polynucleotide, a polypeptide, a polysaccharide, a natural rubber, a polyphenolic polymer, a complex of polymers of large chain fatty acids or their mixtures or their composites.

In one embodiment, the hydrocolloid binding agent is a natural polymer derivative hydrogel. Preferably, the hydrocolloid binding agent is selected from the group of sodium carboxymethyl cellulose (CMC), carboxyethyl cellulose (CEC), hydroxypropyl cellulose (HPC), hydroxyethyl cellulose (HEC), methyl cellulose (MC), ethyl cellulose (EC), hydroxypropylmethyl cellulose (HPMC), hydroxyethylmethyl cellulose (HEMC), hydroxyethylpropyl cellulose (HEPC), bacterial cellulose (BC), cellulose nanofiber (CNF), cellulose nanocrystals (CNC), microfibrillated cellulose (MFC), hydroxypropyl methylcellulose phthalate, or their mixtures.

In one embodiment, the hydrocolloid binding agent is carboxymethyl cellulose or sodium carboxymethyl cellulose.

In one embodiment, the binding agent is present in a concentration between 0.1 and 6% weight (wt.) of hydrogel before drying, preferably between 0.5 and 5% wt., more preferably between 1 and 4% wt.

In one embodiment, the gelling agent from the family of polysaccharide hydrocolloids in the hydrogel formulation for the hydrogel coating layer (1) is selected from, but not limited to, sodium alginate, gellan gum, carrageenan, guar gum, xanthan gum, locust bean gum, gum arabic, pectin, or modified starch. In one embodiment, the gelling agent is present in concentration between 0.1 and 6% wt. of hydrogel before drying, preferably between 0.5 and 5% wt., more preferably between 1 and 4% wt.

In one embodiment, the plasticizer in the hydrogel formulation for the hydrogel coating layer (1) is selected from, but not limited to, polyvinyl alcohol (PVOH), ethylene glycol (EG), diethylene glycol (DEG), triethylene glycol (TEG), polyethylene glycol (PEG), propylene glycol (PG), glycerol, erythritol, sorbitol, mannitol, maltitol, xylitol, polyols, fatty acids, or vegetal oils. In one embodiment, the plasticizer is present in a concentration between 1 and 15% wt. of hydrogel before drying, preferably 2 and 12% wt., more preferably 3 and 8% wt.

The hydrogels, as described above, are formed through a chemical or physical cross-linking of individual polymer chains. The physical cross-linking can be achieved when the polymer is combined with ionic salts composed of an anion (di or trivalent) and a cation (di or trivalent). Chemical cross-linking can be achieved by the formation of intermolecular or intramolecular covalent bonds, where different cross-linkers can be used, as it will be understood by a person skilled in the art.

The hydrogel formulation can be dropcasted, coated, or wet laminated in the cellulosic fibrous layer (2) to produce the hydrogel coating layer (1). The hydrogel formulation can be applied to the cellulosic fibrous layer (2) using several different coating techniques. Examples of these techniques include but are not limited to, rod, grooved rod, curtain, blade, slot-die, applicator roll, fountain, jet, short dwell, slotted die, bent blade, bevel blade, air knife, bar, gravure, size press, spray or any other suitable technique that allows the formation of a smooth coated surface.

Having the cellulosic fibrous layer (2) with the hydrogel formulation provides a layer that allows the hydrogel to be polymerized outside/above the cellulosic fibrous layer (2).

### The Cellulosic Fibrous Layer (2):

The cellulose-based microbiological culture device comprises a cellulosic fibrous layer (2) comprising cellulose fibers with weight in the range of 50 to 500 g/m². The cellulose fibers can be selected from different sources, including but not limited to, bleached softwood fibers, unbleached softwood fibers, bleached hardwood fibers, unbleached hardwood fibers, cotton fibers, or any other suitable fiber source known for a person skilled in the art or a mixture thereof or their mixtures. Additionally, different processes can be used to obtain said fibers, including but not limited to sulfite pulping, kraft pulping, general chemical pulping, mechanical pulping, chemi-mechanical pulping or any other pulping process known to a person skilled in the art.

In one embodiment the cellulosic fibrous layer (2) is obtained from refining the fibers until the desired Schopper Riegler freeness degree (° SR). Refined fibers have a ° SR between 15 and 40. Fibers with different Schopper Riegler freeness degree can be combined to obtain a cellulosic fibrous layer with the appropriated water retention capability. Fiber refining can be done in low consistency, medium consistency or high consistency using processes known to a person skilled in art, including but not limited to conical refiners or disc refiners.

At least one cellulosic fibrous layer (2) comprises a cross-linker agent. The cellulosic fibrous layer (2) comprising the cross-linker is preferably the layer directly in contact with the hydrogel coating layer (1).

The cellulosic fibrous layer (2) is used as substrate for the hydrogel coating layer (1) but also as a cross-linker diffusion matrix allowing gelation to occur only at the time of contact between the polymers and the substrate, this allows the hydrogel formulation to be viable for a much longer period, since cross-linking will not occur immediately in the mixing process of the components of the device. The second advantage is achieving a homogeneous distribution of the cross-linker along the cellulosic fibrous layer (2). The third advantage is that it prevents all the hydrogel from entering the cellulosic fibrous layer (2) as the gelling will occur on the two layers interface, allowing the vast majority of the cellulosic fibrous layer (2) volume to remain with the original structure while maintaining a high rehydration speed and water retention capacity.

In one embodiment the cross-linker agent improves hydrogel strength and is selected from, but not limited to, zinc chloride, magnesium chloride, copper sulfate, nickel sulfate, calcium chloride, epichlorohydrin, or citric acid. In one embodiment, the cross-linker agent is present between 0.01 and 5% wt. of hydrogel before drying, preferably between 0.05 and 2% wt., more preferably between 0.1 and 1% wt.

In one embodiment, the cellulosic fibrous layer (2) can comprise a culture medium. In this embodiment, the culture medium is impregnated in this layer.

In another embodiment, the culture medium is added to the cellulose-based microbiological culture device before rehydration.

Any culture medium known in the field can be used in the cellulosic fibrous layer (2) in an amount suitable to carry out microbial growth in the device.

In one embodiment, the cellulosic fibrous layer (2) contains a wet strength agent selected from, but not limited to, polyaminopolyamide-epichlorohydrin resins, glyoxalated polyacrylamide resins, polyvinylamine resins, polyethylenimine resins, polyisocyanate resins, dialdehyde starch, or a mixture thereof.

In one embodiment, the cellulosic fibrous layer (2) may further comprise other materials, such as a colorimetric agent, i.e. chromophores, or cell growth markers.

In one embodiment, the cellulose-based microbiological culture device comprises at least two stacked cellulosic fibrous layers (2) allowing to increase the water retention capabilities. In this embodiment, the lower cellulosic fibrous layer (2), which is not in direct contact with the hydrogel coating layer (1), is arranged and bonded below the upper cellulosic fibrous layer. This second cellulosic fibrous layer (2) is designed to have rehydration means (3) allowing to hydrate the device from the bottom cellulosic fibrous layer (2).

### Rehydration Means (3):

In one embodiment, the rehydration means (3) is an additional layer arranged below the cellulosic fibrous layer (2).

In one embodiment the rehydration means (3) are selected from a perforation in the hydrogel coating layer (1) and the cellulosic fibrous layer (2), a perforation on the hydrogel coating layer (1), or a lateral access tab via one hydration coating layer (1) as shown in FIG. 2, or a rehydration tab, micro perforation of selected areas of the device, or a single use blister.

More specifically, in one embodiment, the hydration of the cellulose-based microbiological culture device is made via a single use water blister connected to at least one cellulosic fibrous layer (2), in a preferred embodiment the bottom layer (2), that allows for a fast and homogeneous dispersion of water, reducing the need for external equipment.

In one embodiment, the hydrogel coating layer (1) can be separated from the cellulosic fibrous layer (2), allowing for easier visual detection, for example via light transmittance, and/or for further contact reinoculation onto specific media, or further sample processing for molecular characterization methods.

In one embodiment, a double-sided adhesive material is arranged on the underside surface of the cellulosic fibrous layer (2). The resulting cellulose-based microbiological culture device can be attached to a surface, such as a petri dish or other suitable surface, to allow the inverted incubation of the device.

### Antibiogram

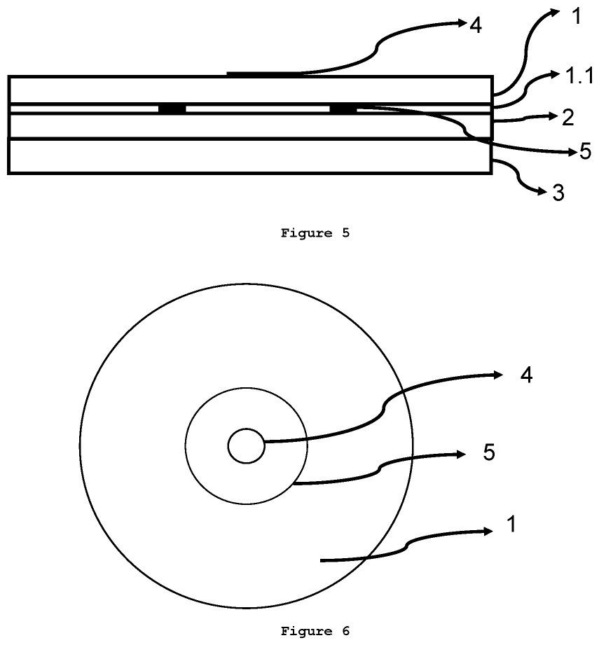

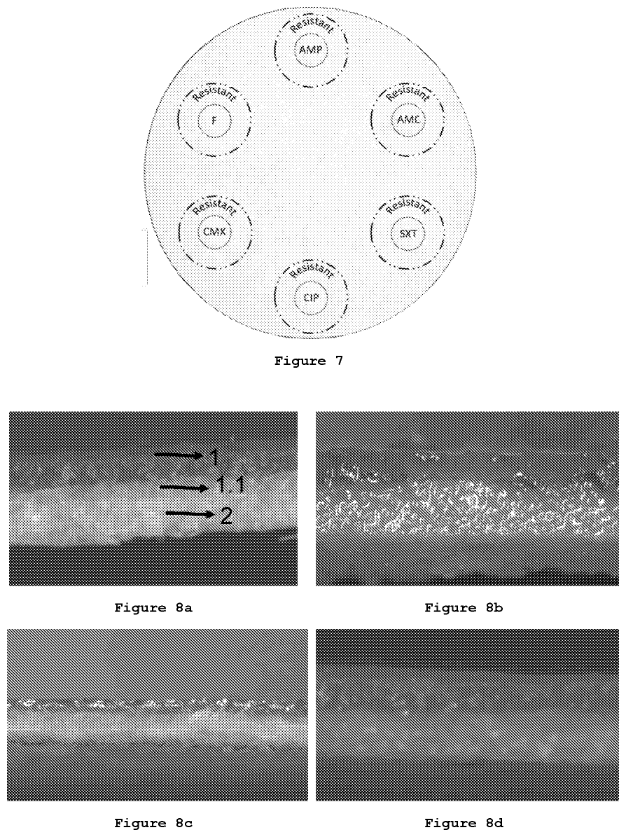

In one embodiment, the cellulose-based microbiological culture device comprises an antibiogram printed on the cellulosic fibrous layer for drug susceptibility testing and colorimetric detection as shown in FIGS. 5 to 7. Working with an integrated culture media suppresses the need of a separate culture dish for this test, reducing waste material and analysis time, while providing a visual color-based direct result.

The incorporation of antibiograms in the disclosed cellulose-based microbiological culture device is done by directly printing (for example by inkjet or screen printing) at least one antibiotic solution (4) in the hydrogel coating layer (1) coincident with at least one threshold halo (5). This application is innovative in concept and production process, allowing to reduce the analysis time. The integrated test comprises at least one threshold halo (5) printed on the cellulosic fibrous layer (2) for each antibiotic, making it easier to score each plate without any external equipment.

### E-Test

The proposed “E-test” solution will be the first to integrate the antibiotic gradient (6), colorimetric detection, and culture media in one device as shown in FIG. 11. Working with an integrated culture media suppresses the need of a separate culture dish for this test, reducing waste material and analysis time, while providing a visual color-based direct result. A ruler like structure (7) is arranged in the cellulosic fibrous layer (2) to facilitate the scoring of samples. An antibiotic gradient (6) is added to the hydrogel coating layer (1) and is aligned with the ruler like structure (7).

In one embodiment, at least one antibiotic gradient (6) is deposited by screen-printing in a carboxymethylcellulose-based ink. The step of applying a hydrogel formulation in the cellulosic fibrous layer (2) forming at least a layer zone comprising an antibiotic gradient may be carried out in such a way to form a pattern in specific regions of the hydrogel layer (1).

In another embodiment the cellulose-based microbiological culture device can be used by placing the device upside down (i.e., with the hydrogel layer (1) facing downwards) in a transparent container. After hydration the cellulosic fibrous layer (2) can be removed from the hydrogel layer (1) before inoculation. This approach allows for the use of a fully transparent growth matrix with all the previous described characteristics.

### Method

The present invention also relates to a method of producing the cellulose-based microbiological culture device comprising the following steps:
impregnating a cross-linker agent in a cellulosic fibrous layer (2);
applying a hydrogel formulation in the cellulosic fibrous layer (2) forming at least one hydrogel layer (1);
drying the device at a temperature between 20 to 50° C.;
Optionally, adding a culture medium solution to the cellulosic fibrous layer (2) and drying the device at a temperature between 20 to 50° C.

In one embodiment after applying the hydrogel formulation, the device can be washed with water for a period of time of less than 40 min and then dried at a temperature between 20 to 50° C.

The applying step is repeated according to the number of hydrogel coating layers (1) desired.

For devices with more than one cellulosic fibrous layer (2), the method further comprises a step of stacking and bonding at least two cellulosic fibrous layer (2) on top of each other.

In one embodiment, the method further comprises the steps of printing at least one antibiotic solution in the hydrogel coating layer (1) and printing at least one threshold halo on the fibrous cellulosic layer (2) for each antibiotic, to obtain an antibiogram.

In one embodiment, the method further comprises the steps of printing at least one antibiotic gradient in the hydrogel coating layer (1) comprising a colorimetric agent, to obtain an E-test.

The hydrogel formulation comprises at least one binding agent from the family of cellulose-derived hydrocolloids, at least one gelling agent from the family of polysaccharide hydrocolloids and a plasticizer, as described above.

The device is dried with hot air or infra-red lamps or any other suitable technique to dehydrate the hydrogel. Alternately to hot air drying, the hydrogel can also be lyophilized.

The method according to the present invention also enables the individualization of the hydrogel coating layer (1), as the hydrogel formulation can be deposited only at selected and defined regions of the cellulosic fibrous layer (2). This procedure allows localized antibiotic rich regions on the hydrogel coating layer (1), allowing for antibiotic susceptibility testing. Moreover, the antibiotic can be deposited in controlled amounts at defined regions allowing the production of antibiotic gradients over the hydrogel coating layer (1), hence creating a device that can determine the MIC of microorganisms (such as E-test).

The embodiment of the present invention comprising an array of layer zones impregnated with an antibiotic can be combined with guide markers printed on the cellulosic fibrous layer (2) providing an easy score of susceptibility.

Once produced, the cellulose-based microbiological culture device can be arranged on a support selected from a petri dish, an impermeable cellulosic support or any other suitable support for the device.

The present invention further relates to the use of the cellulose-based microbiological culture for device the culture, detection, characterization, identification, and enumeration of microorganisms. This device is also suitable, but not limited, to be used with biological samples, for example urine, food and beverage samples, or environmental samples.

The present invention can be used for contact inoculation by contact with a surface.

Surface monitoring methods are used to evaluate the effectiveness of hygiene procedures and overall cleanliness of surfaces. Surface monitoring tests use contact plates or swabs according to ISO standards. The swab method is suitable for uneven surfaces; while contact plates only allow for smooth surfaces. However, contact plates allow for consistent and quantitative contamination results. Additionally, swabs can cover larger areas when compared to contact plates. The proposed technology is suitable to be used both via contact inoculation and swab methods; allowing for a wide range of application scenarios without the use of extra disposable materials. The present invention can be used as a standard contact plate using standard protocols. When pressed on the test area, the exposed media forms an impression of the surface collecting any viable microorganisms that may be present. Once analyzed, the level of growth is reported as colony-forming units (CFU). Comparing the level of growth pre and post sanitization will provide insight into the efficacy of sanitization or disinfection programs. Because not all surfaces are flat and/or smooth, surface swabbing can also be implemented. The fibrous cellulosic material and the hydrogel structure allows this technology to be used to directly swab the surface without deformation or degradation of the growth media structure. Moreover, a controlled amount of water can be added to the test area allowing for the inoculation and hydration of the media to occur in one single step while increasing the efficiency of sample collection. This approach creates the advantage of a single test for multiple surface types and coverage areas in a single product.

The present invention can also be used for the analysis, selection and optimization of microorganisms that metabolize cellulose that can have applications in recycling or reuse of cellulose for energetic purposes.

Lignocellulose is one of the most abundant organic materials in the biosphere. Knowledge of cellulose-degrading microbial taxa is of significant importance with respect to nutrition, biodegradation, biotechnology, and the ability to understand/control the largest flow of carbon in the ecosystem. The detection, cultivation, and isolation of such cellulose metabolizing organisms from complex microbial populations and environments is still a challenge. To overcome this, there is the need to develop technologies for the screening, isolation, and cultivation of cellulolytic microorganisms from the environment. The presently disclosed invention is built as a cellulosic solid growth substrate. This invention provides fast and direct screening assays for the direct enumeration and isolation of cellulase-producing organisms from natural materials, and for the selection of cellulolytic mutants of bacteria and fungi. This method enabled estimates of cellulolytic bacterial counts, confirmed the cellulolytic activities of pure cultures and allowed the morphological characteristics and other properties of cellulolytic bacteria to be determined. The formation of matrix degradation halos and transparent zones around colonies on the presently disclosed device unequivocally indicates cellulose decomposition. Results can be analyzed via the ratio of colony size versus the hallo formed in the solid matrix via cellulose degradation (as shown in FIGS. 10a and 10b). Additionally, this technology can be integrated with colorimetric indicators (e.g. Congo Red) allowing to have a colorimetric detection and easy observation of cellulose degradation and quantification.

### Results

FIGS. 8a to 8d show the cross-sections of the present invention with different ionic cross linkers, when dry and after hydration. This experiment shows that the proposed material can undergo hydration cycles with structural integrity.

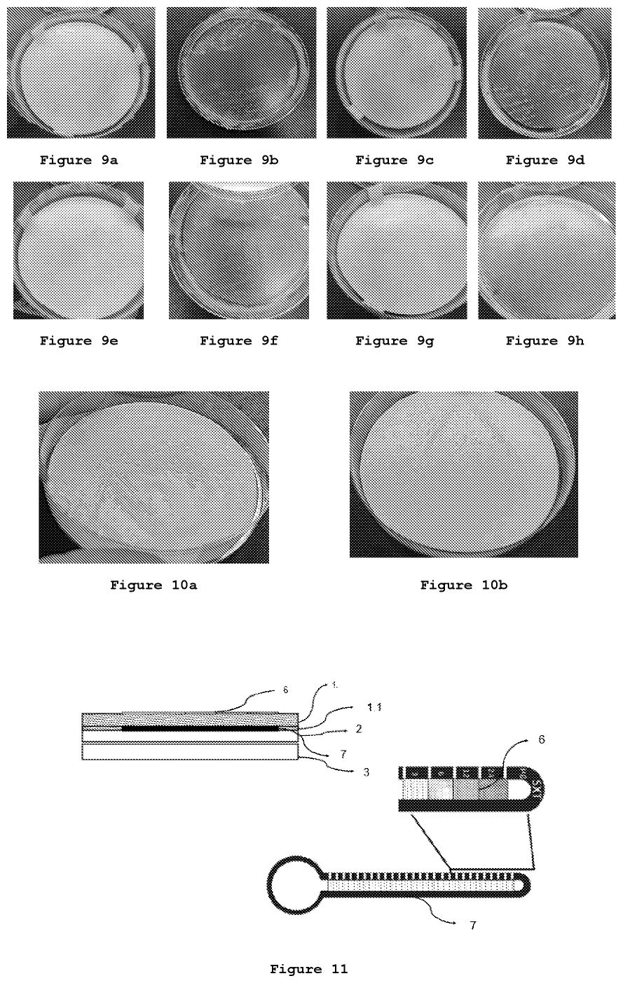

FIGS. 9a to 9h show the present invention with two different culture media and growing E. coli and S. Aureus.

No growth was observed in the case of S. Aureus in MacConkey medium since this medium is gram+ selective. This experiment shows that the proposed technology allows for bacterial growth and colony isolation with macroscopic and morphologic characteristics comparable to those of standard methods.

FIGS. 9a to 9d shows the present invention with two culture media and growing E. coli and S. Aureus.

Additionally, these results show the use of selective media (e.g., MacConkey) allowing for specific gram differentiation/detection. No growth was observed in the case of S. Aureus in MacConkey medium since this medium is gram+ selective.

1. A cellulose-based microbiological culture device comprising:
a dehydrated hydrogel coating layer arranged on top of or partially integrated on at least one cellulosic fibrous layer; and
at least one rehydration means;
wherein the dehydrated hydrogel coating layer comprises:
at least one binding agent from the family of cellulose-derived hydrocolloids in a concentration between 0.1 and 6% wt.;
at least one gelling agent from the family of polysaccharide hydrocolloids in a concentration between 0.1 and 6% wt.; and
a plasticizer in a concentration between 1 and 15% wt.;
and wherein the at least one cellulosic fibrous layer further comprises a cross-linker agent in a concentration between 0.01% to 5% wt.

2. The cellulose-based microbiological culture device according to claim 1, wherein the device comprises an interface formed by the hydrogel layer being partially integrated in the cellulosic fibrous layer.

3. The cellulose-based microbiological culture device according to claim 1, wherein the dehydrated hydrogel coating layer has a thickness between 5 and 500 μm.

4. The cellulose-based microbiological culture device according to claim 1, wherein the device comprises at least two stacked cellulosic fibrous layers.

5. The cellulose-based microbiological culture device according to claim 1, wherein the hydrocolloid binding agent is selected from the group consisting of cellulose and/or a cellulose derivative, a polynucleotide, a polypeptide, a polysaccharide, a natural rubber, a polyphenolic polymer, a complex of polymers of large chain fatty acids, mixtures and composites thereof.

6. The cellulose-based microbiological culture device according to claim 1, wherein the hydrocolloid binding agent is selected from the group consisting of sodium carboxymethyl cellulose (CMC), carboxyethyl cellulose (CEC), hydroxypropyl cellulose (HPC), hydroxyethyl cellulose (HEC), methyl cellulose (MC), ethyl cellulose (EC), hydroxypropylmethyl cellulose (HPMC), hydroxyethylmethyl cellulose (HEMC), hydroxyethylpropyl cellulose (HEPC), bacterial cellulose (BC), cellulose nanofiber (CNF), cellulose nanocrystals (CNC), microfibrillated cellulose (MFC), hydroxypropyl methylcellulose phthalate, and mixtures thereof.

7. The cellulose-based microbiological culture device according to claim 1, wherein the gelling agent from the family of polysaccharide hydrocolloids is selected from the group consisting of sodium alginate, gellan gum, carrageenan, guar gum, xanthan gum, locust bean gum, gum arabic, pectin, and modified starch.

8. The cellulose-based microbiological culture device according to claim 1, wherein the plasticizer is selected from the group consisting of polyvinyl alcohol (PVOH), ethylene glycol (EG), diethylene glycol (DEG), triethylene glycol (TEG), polyethylene glycol (PEG), propylene glycol (PG), glycerol, erythritol, sorbitol, mannitol, maltitol, xylitol, polyols, fatty acids, and vegetal oils.

9. The cellulose-based microbiological culture device according to claim 1, wherein the cellulosic fibrous layer comprises cellulose fibers with weight in the range of 50 to 500 g/m², selected from the group consisting of bleached softwood fibers, unbleached softwood fibers, bleached hardwood fibers, unbleached hardwood fibers, cotton fibers and mixtures thereof.

10. The cellulose-based microbiological culture device according to claim 1, wherein the cross-linker agent is selected from the group consisting of zinc chloride, magnesium chloride, copper sulfate, nickel sulfate, calcium chloride, epichlorohydrin, and citric acid.

11. The cellulose-based microbiological culture device according to claim 1, wherein the cellulosic fibrous layer further comprises a culture medium.

12. The cellulose-based microbiological culture device according to claim 1, wherein the cellulosic fibrous layer further comprises a wet strength agent selected from the group consisting of polyaminopolyamide epichlorohydrin resins, glyoxalated polyacrylamide resins, polyvinylamine resins, polyethylenimine resins, polyisocyanate resins, dialdehyde starch, and a mixture thereof.

13. The cellulose-based microbiological culture device according to claim 1, wherein the cellulosic fibrous layer further comprises a colorimetric agent or cell growth markers.

14. The cellulose-based microbiological culture device according to claim 1, wherein the at least one rehydration means is selected from the group consisting of a layer arranged below the cellulosic fibrous layer, a perforation in the hydrogel coating layer and the cellulosic fibrous layer, a perforation on the hydrogel coating layer, a lateral access tab via one hydration coating layer, a rehydration tab, micro perforation of selected areas of the device, and a single use blister.

15. The cellulose-based microbiological culture device according to claim 1, wherein the cellulosic fibrous layer comprises at least one threshold halo for an antibiotic and the hydrogel coating layer comprises at least one antibiotic solution coincident with the least one threshold halo forming an antibiogram.

16. The cellulose-based microbiological culture device according to claim 1, wherein the cellulosic fibrous layer comprises a ruler like structure and the hydrogel coating layer comprises antibiotic gradient aligned with the ruler like structure, forming an E-test.

17. The cellulose-based microbiological culture device according to claim 1, wherein a double-sided adhesive material is arranged on the underside surface of the cellulosic fibrous layer.

18. A method of producing the cellulose-based microbiological culture device described in claim 1 comprising the following steps:
impregnating a cross-linker agent in a cellulosic fibrous layer;
applying a hydrogel formulation in the cellulosic fibrous layer forming at least one hydrogel layer;
drying the device at a temperature between 20 to 50° C.; and
optionally, adding a culture medium solution to the cellulosic fibrous layer and drying the device at a temperature between 20 to 50° C.

19. The method according to the claim 18, wherein the applying the hydrogel formulation step is repeated according to the number of hydrogel coating layers desired.

20. The method according to claim 18, further comprising a step of stacking and bonding at least two cellulosic fibrous layer on top of each other.

21. The method according to claim 18, further comprising the steps of printing at least one area with an antibiotic solution in the hydrogel coating layer and printing at least one threshold halo on the fibrous cellulosic layer for at least one antibiotic.

22. The method according to claim 18, further comprising the steps printing at least one area with at least one antibiotic gradient in the hydrogel coating layer.

23. A method for culture, detection, characterization, identification, and enumeration of microorganisms comprising using the cellulose-based microbiological culture device described in claim 1 to culture, detect, characterize, identify and enumerate organisms.
